(19) United States
(12) Patent Application Publication
(10) Pub. No.: US 2015/0374882 A1
(43) Pub. Date: Dec. 31, 2015

(54) Porous material

(71) Applicants: Robert Anthony McDemus, Washington, PA (US); Martha L. Serafin, Lakeport, MI (US)

(72) Inventors: Robert Anthony McDemus, Washington, PA (US); Louis A. Serafin, JR., Lakeport, MI (US)

(21) Appl. No.: 14/545,785

(22) Filed: Jun. 18, 2015

Related U.S. Application Data
(60) Provisional application No. 61/998,178, filed on Jun. 20, 2014.

(51) Int. Cl. A61L 27/56; C23C 4/18; C23C 4/06; A61L 27/30; C23C 4/12

(52) U.S. Cl. CPC A61L 27/56; A61L 27/306; C23C 4/128; C23C 4/06; C23C 4/18

(57) ABSTRACT

Porous material made with metal or metal alloy, which may be a coating, can be made from a first intermediate and then a second intermediate. For instance, the first intermediate may be provided by providing the metal or metal alloy, and a pore-holding substance; and combining the metal or metal alloy, and the pore-holding substance. The second intermediate can be provided, for instance, by forming from the first intermediate, a matrix of metal or metal alloy, in which is dispersed the pore-holding substance, for example, with thermal spraying. The porous material can be made by contacting the second intermediate with a pore-forming substance under conditions such that pore-holding substance in the matrix contacted with the pore-forming substance is reduced in size or removed to leave pores in remaining metal or metal alloy to provide a constituent of a metal or metal alloy with pores.

This claims benefit under 35 USC 119(e) of provisional application No. U.S. 61/998,178 filed on Jun. 20, 2014 A.D. The specification of that application, to include its drawings, is incorporated herein by reference in its entirety.

### FIELD AND PURVIEW OF THE INVENTION

This concerns a porous material made with metal or metal alloy, an intermediate therefor, and methods of making the porous material and intermediate, and uses thereof. The porous material, as an example, may be a coating as part of an orthopedic implant.

### BACKGROUND TO THE INVENTION

Porous materials made of metals or metal alloys are known to provide various benefits. As an example, in orthopedic implants, porous coatings can assist in fixation of the implants by bone ingrowth into the coatings. Roughened or grit-blasted surfaces may have less effect.

Various metals or metal alloys can be employed to make porous coatings. In the orthopedic implant art, these can include, for example, tantalum or titanium. Also, hydroxylapatite (also called hydroxyapatite) and other forms of calcium phosphate are examples of materials used for coating orthopedic implants. As substrates for the coatings, metals and certain ceramics, especially now magnesium oxide stabilized tetragonally toughened zirconia (MgO-TTZ), are known. See, e.g., Pub. Nos. US 2006/0025866 A1 of Serafin, Jr. et al., US 2010/0076566 A1 of Serafm, Jr. et al., and US 2013/0131824 of Meehan et al. Compare, Pub. No. US 2013/0248847 A1 of Mayfield et al.

To make known porous coatings of metal, various techniques can be employed. Among these are various forms of thermal spray, vapor metal deposition, sputtering, brazing, and sintering. A powder such as a soluble calcium compound may be blasted at the coating. Nitric acid treatment dissolves that compound and passivates the implant coating. A potassium hydroxide treatment, grit blasting with calcium phosphate, nitric acid passivating, and so forth can be used for a nanoscale roughened surface. Compare, Meehan et al., Mayfield et al.

Among difficulties include provision of a reliable, suitable pore size distribution, especially to the rigorous standards of certifying bodies as, for example, in the United States, the Food and Drug Administration. Another consideration is manufacturing cost or efficiency.

It would be desirable to improve the art. It would be desirable to ameliorate if not substantially solve or improve one or more problems or difficulties in the art. It would be desirable, more particularly, to improve efficiencies at which porous coatings with metal can provided both in reliability of suitable pore size distributions and in manufacturing costs or efficiencies. It would be desirable to provide the art an alternative.

### A DISCLOSURE OF THE INVENTION

Provided hereby is a porous material made with metal or metal alloy, which comprises a constituent of a metal or metal alloy, which, for example, when embodied as a porous coating on an orthopedic implant, has a volume porosity about from 30 to 70 percent, an average pore size about from 100 to 1000 microns, interconnecting porosity, and a thickness when embodied as a porous coating about from 500 to 1500 microns, and other characteristics, which, taken together, distinguishes it from other porous materials and/or porous coatings known in the art; optionally with embedded pore-holding material internal to the constituent. When residing on a substrate, for example, the porous material can exist as a coating. Provided also is a first intermediate, say, by a method comprising providing a metal or metal alloy, and a pore-holding substance; and combining the metal or metal alloy, and the pore-holding substance. A second intermediate can be provided, say, by forming from the first intermediate, a matrix of metal or metal alloy, in which is dispersed the pore-holding substance, for example, with thermal spraying. A porous material made with the metal or metal alloy can be made by contacting the second intermediate with a pore-forming substance under conditions such that pore-holding substance in the matrix contacted with the pore-forming substance is reduced in size or removed to leave pores in remaining metal or metal alloy to provide a constituent of a metal or metal alloy with pores.

The invention is useful in providing a porous material made with metal or metal alloy.

Significantly, by the invention, the art is advanced in kind, and the art is provided an alternative. It ameliorates if not substantially solves or improves one or more problems or difficulties in the art, to include superior bond strength when embodied as a coating, and abrasion resistance. Additionally it provides superior control of the percent porosity and the pore size of the porosity the porous material. It improves efficiencies at which porous materials, to include coatings, with metal can provided both in reliability of suitable pore size distributions and in manufacturing costs or efficiencies. The second intermediate has a matrix of a metal or metal alloy combined with a soluble material that will be dissolved after it has been sprayed, which is especially useful for a coating, notably for an orthopedic implant. The volume of the soluble material can be altered to control the percentage of the porosity and the average pore size of the porosity of the porous material as a final product. Thus, the invention provides for better control of the porosity size and distribution while maintaining mechanical properties and abrasion resistance, and not only that, a convenient and reliable way of achieving the same.

Numerous further advantages attend the invention.

The drawings form part of the specification hereof. With respect to the drawings, which are not necessarily drawn to scale, the following is briefly noted:

FIG. 1 is a cross-sectional view of a first intermediate, embodied as an isolated powder.

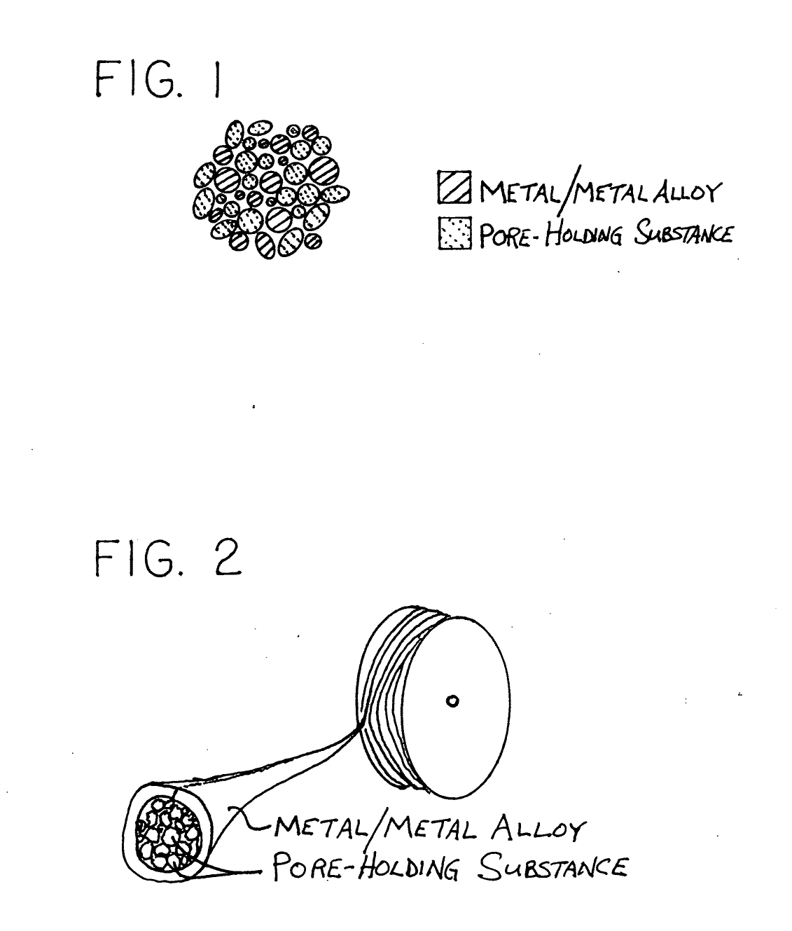

FIG. 2 is a view of another first intermediate, embodied as a cored wire.

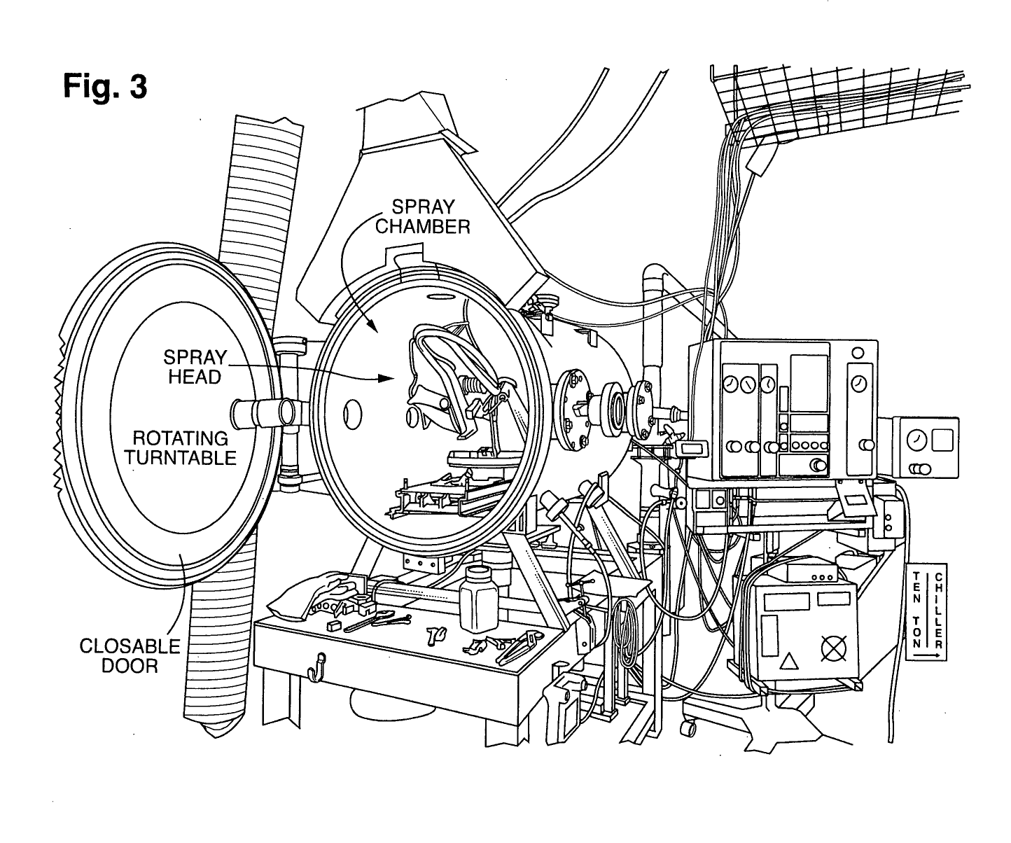

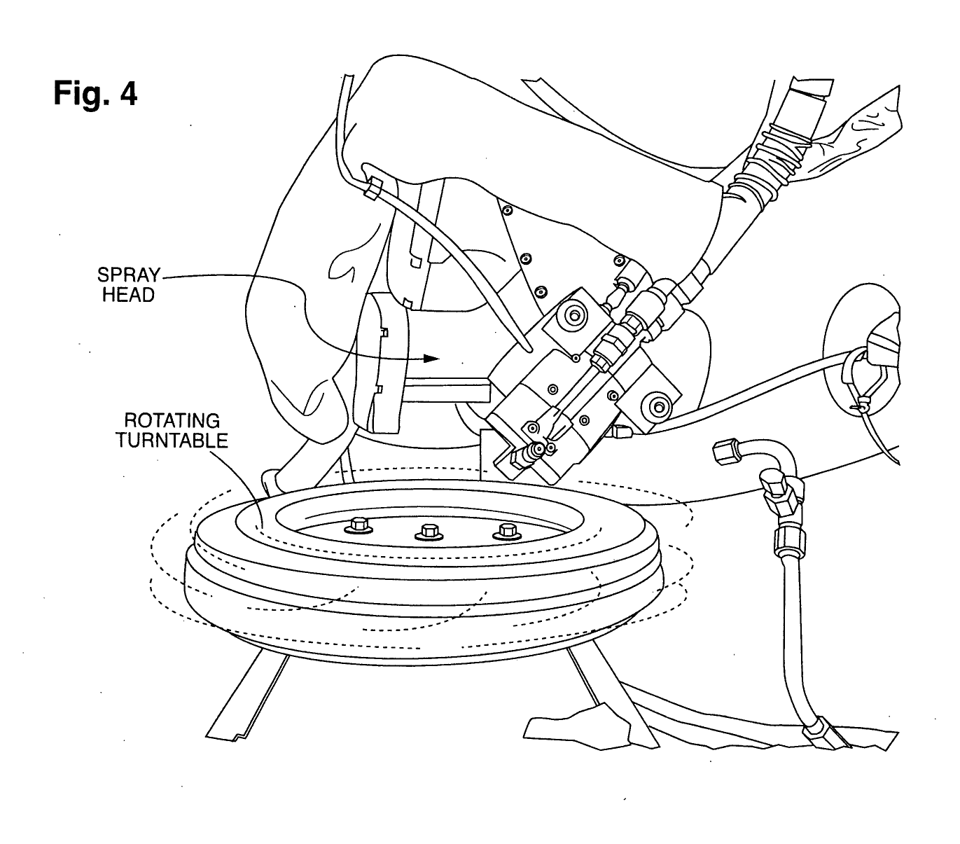

FIGS. 3 and 4 are views of thermal spray equipment that can be used to make a second intermediate, with immediate or in situ use of a first intermediate.

FIG. 5 is a cross-sectional view showing an embodiment of a second intermediate, for example, as made with thermal spray equipment as of FIGS. 3 and 4.

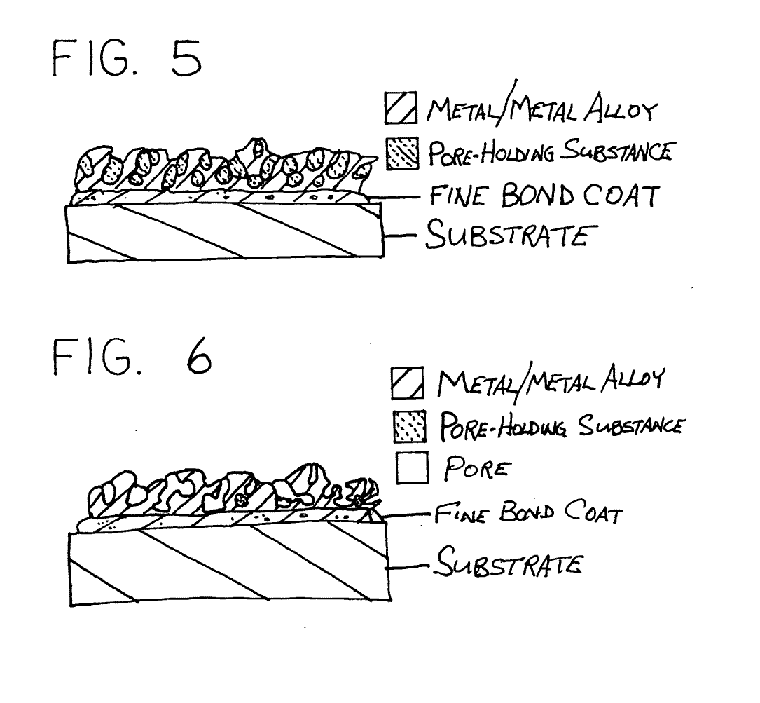

FIG. 6 is a cross-sectional view showing an embodiment of a porous material, for example, as made from passivation of a second intermediate as of FIG. 5.

The invention can be further understood by the following detail, which may be read in view of the drawings. As with the foregoing disclosure, the following disclosure is to be read in an illustrative, but not necessarily limiting, sense.

Hereby, a porous material is made with metal or metal alloy after a soluble or otherwise removable or reducible-in-size part of its precursor, the second intermediate, is dissolved, otherwise removed or reduced in size. The porous material can reside on, which can include being mechanically, chemically and/or metallurgically bonded to, a substrate, for example, as a coating; or it can be independent from a substrate. Among substrates may be mentioned metals such as of aluminum, chromium, cobalt, iron, nickel, tantalum, titanium, vanadium, zirconium, and even such metals as gold, silver, platinum, and so on; metal alloys such as those including cobalt, chromium, nickel, iron, molybdenum titanium, aluminum, vanadium, and so forth, say, a cobalt-chromium alloy, a titanium-6-vanadium-4-aluminum alloy, a stainless steel, and so on; and suitable ceramics, which may include titanium nitride, various zirconia ceramics, to include a magnesium oxide stabilized zirconia, especially MgO-TTZ, and so on; and so forth and the like. The porous material may exist away from a substrate, i.e., may not exist as a coating, which may be brought about by making the porous material apart from a substrate, say, in a thermal spray process in a chamber, or by making it on, and then afterwards removing it from, a substrate, say, by cutting it off the substrate or by impinging it on a substrate from which the porous material can be removed such as, for example, in the case of titanium plasma sprayed on a smooth alumina ceramic surface. The porous material, especially as a coating, can be applied with any suitable thermal spray process, for example, combustion wire, combustion powder, twin wire electric arc, plasma spray, high velocity oxygen fuel (HVOF), or cold spray. The porous material, especially as a coating, can be applied under a vacuum; in a controlled atmosphere, say, with inert gas; in air; at a partial pressure atmosphere; or in air or another gas with a shroud. In these cases, the metal or metal alloy matrix that remains has the desired percent porosity, average pore size, and so forth, for a soluble part of that second intermediate can be removed afterwards.

By definition, the porous material includes a constituent of the metal or metal alloy, which has a surface area; and, in the constituent, pores. The pores may be of any suitable distribution, for instance, in a porous coating for an orthopedic implant, that which has a volume porosity about from 30 to 70 percent, an average pore size about from 100 to 1000 microns, interconnecting porosity, and a thickness when embodied as a porous coating of about from 500 to 1500 microns, and other characteristics, say, tensile strength, shear strength and/or shear fatigue strength, which, when taken together, distinguishes it from other porous coatings known in the art. Other characterizing properties of the porous material can include biocompatibility, inert or passivation coating, and so forth. A medicament may be provided with the porous material, for example, being provided within its pores. An embedded pore-holding substance may be internal to the constituent.

The porous material is made in steps. One intermediate or more may precede it. An intermediate may be employed immediately, as it were in situ, or isolated for later processing.

A method to make a first intermediate for a porous material made with metal or metal alloy is thus provided. It includes providing a metal or metal alloy, and a pore-holding substance. The metal or metal alloy can be selected from those mentioned above. It may be in a form of a solid such as sample of a powder or rod-like slivers and/or other form. Alternatively, it may be in a form of a sheath or hollow wire, in which can be deposited, or which can be wrapped around, the pore-holding substance. The pore-holding substance is a substance that, when present in a matrix of solid metal or metal alloy and contacted with a pore-forming substance, is reduced in size or removed to leave pore(s) in the solid metal or metal alloy in which it had been present. The pore-holding substance in the first intermediate may be in a form of a solid such as sample of a powder or rod-like slivers and/or other form. Examples of pore-holding substances include calcium phosphate, which may be hydroxyapatite or another calcium phosphate; dicalcium phosphate; tricalcium phosphate; magnesium oxide; or, in general, anything leachable or otherwise able to be removed, dissolved, sublimed, melted, reacted, or induced to undergo a reduction of occupied volume in the solid metal matrix with contact of the pore-forming substance. For orthopedic implant applications, a calcium-based or other pore-holding substance may be employed to engender bone-ingrowth, especially if pore-holding substance would remain in the porous material of the implant. For example, in a first intermediate, an apatitic calcium phosphate abrasive powder may be provided for the pore-holding substance, with commercially pure titanium (CPT) powder as the metal or metal alloy. Additional material(s) may be present as a pore-holding substance.

A powder herein embraces solid matter in a finely divided state or so-called particulate matter. The powder may generally have a substantial if not major or even predominant amount of particles that are substantially regular in appearance such as generally spherical or rounded, generally ellipsoid or football, generally cubic or shoebox shaped, and so forth, or irregular in appearance yet not generally substantially elongate when the sample of powder is considered as a whole, in contrast to rod-like slivers. However, for example, should a powder sample include rod-like slivers, it can be referred to more generically as “particulate matter.” Although any suitable size or size distribution powder sample may be employed, some examples of powder sizes or powder size samples that may be employed may be as follows:

Metal or metal alloy: about from 35 to 500 um (+35/−400 mesh);
about from 44 to 150 um (+100/−325 mesh);
about from 75 to 175 um (+80/−200 mesh).

Pore-holding substance: about from 35 to 500 um (+35/−400 mesh);
about from 53 to 350 um (+45/−270 mesh);
about from 63 to 106 um (+140/−230 mesh);
about from 150 um to 200 um (+70/−100 mesh);
about from 180 to 360 um (+45/−80 mesh);
about from 180 to 300 um (+50/−80 mesh);
about 180 um or less (+80 mesh).

The metal or metal alloy, and the pore-holding substance are mixed. The mixing may be carried out manually, for example, by stirring a powdered pore-forming substance in with a powdered or liquid metal or metal alloy; or mechanically, for example, by feeding two or more powder samples at the same time into a thermal spray torch using two or more different feeding systems such as powder feeders. Mixing the metal or metal alloy and the pore-holding substance, in general, provides the first intermediate, which may be immediately transformed further, temporarily held or more permanently packaged and stored, and perhaps transported and/or sold, for later transformation. Alternatively, as alluded to above, a suitable metal or metal alloy sheet may be wrapped around the pore-holding substance such as in powder and/or rod-like sliver form to make cored wire, which is a form of the first intermediate.

A second intermediate can be made by forming, for example, from the first intermediate, a matrix of the metal or metal alloy, in which is dispersed the pore-holding substance. The second intermediate may be considered to be made without isolatable formation of a first intermediate such as in cases such as where mixing of a powdered metal or metal alloy and a powdered pore-forming substance is carried out at a thermal spray head, which is immediately or simultaneously sprayed to make the matrix of the metal or metal alloy, in which is dispersed the pore-holding substance. With thermal spraying, for example, the metal or metal alloy and/or the pore-holding substance may melt in whole or in in part, for instance, perhaps with a calcium phosphate and CPT powder couple. Solidification may occur with cooling. On the other hand, a pore-holding substance may have a higher melting point than the metal or metal alloy employed in a thermal spray technique, and not melt, for instance, perhaps with a magnesium oxide and CPT powder couple. The matrix is a solid, and may be considered to constitute a part or the whole of a pre-constituent of the porous material, as it were, from which, along with included pore-holding substance embedded therein, the porous material may be made.

The type of thermal spraying can vary. An electrically generated arc may be employed. A pore-holding material, say, as a powder may be introduced at any suitable manner in thermal spraying. For example, it may be introduced as a powder internally to a spray head, say, with the metal or metal alloy introduced as a powder internally to the spray head. Alternatively, it may be introduced as a powder through one or more jets external to the flame but spraying it into the flame that has metal or metal alloy introduced as a powder internally to the spray head. As another alternative, a cored wire first intermediate can be employed in a thermal spraying technique where the cored wire first intermediate is fed into the spray head for the spraying.

Any suitable ratio of metal or metal alloy to pore-holding material may be employed. As illustrations, a metal or metal alloy:pore-holding ratio by volume may be about from 1:20 to 20:1; 1:10 to 5:1; 1:5 to 2:1; and so forth. Thus, such a ratio may be about 1:1 or 5:2.

Thermal spraying can be conducted in non-atmospheric-pressure conditions. The non-atmospheric conditions may be sub-atmospheric conditions. Such conditions may be carried out or initiated under low vacuum conditions, conditions approaching high vacuum conditions, or high vacuum conditions. Herein, for example, with a vacuum pump reducing a spray chamber atmosphere to a pressure below atmospheric pressure, the following, in general, would apply:

Low vacuum conditions: below about 760 Torr down to about 50 Torr.

Conditions approaching high vacuum conditions: between about 50 and 25 Torr.

High vacuum conditions: about 25 Torr and below.

Such conditions may apply to the partial pressure of a single gas or to a mixture of gases such as air, for example, when air is removed from a spray chamber and replaced with an inert gas. High vacuum conditions may engender a more dense matrix with stronger bonding than atmospheric pressure or low vacuum conditions would. Be that as it may, the spray chamber may have residual air left, the oxygen of which especially should burn off with start of an arc, or it may be flushed with generally inert gas, before or more efficiently after application of any vacuum, for example, Argon, Helium, Nitrogen and/or Hydrogen. The generally inert gas may match gas employed as a feeder and/or shielding gas for the spray head. Initial evacuation of air in a chamber may be carried out, followed by purging and filling the chamber with inert gas to a higher pressure, say, about atmospheric pressure (760 Torr), for thermal spraying.

Any suitable thickness of coating may be applied. For instance, depth of a single coating layer may be about from 0.002 to 0.050 inches. An underlying coat or underlying coats of metal or metal alloy without pore-holding substance can be applied as a bond coat. The bond coat or the substrate itself can have one or more coats of metal or metal alloy with pore-holding substance applied thereto. For example, a 0.003- to 0.008-inch metal or metal alloy bond coat may be applied, on top of which is applied a 0.005- to 0.020-inch coat of metal or metal alloy with pore-holding substance.

The second intermediate may be subsequently transformed, say, after cooling to room temperature, or temporarily held or more permanently packaged and stored, and perhaps transported and/or sold, for later transformation. Transformation can make the porous material.

The porous material made with metal or metal alloy can be made by contacting the second intermediate with a pore-forming substance such that pore-holding substance in the matrix exposed to contact with the pore-forming substance is reduced in size or removed to leave pores in remaining metal or metal alloy, i.e., in the constituent metal or metal alloy. An example of a pore-forming substance is nitric acid, particularly in liquid or properly diluted liquid solution form, say, as an aqueous solution, especially, for example, with a calcium phosphate or magnesium oxide as the pore-holding material. Other acids, and, depending on the pore-holding substance, bases, or other reactive compounds or compositions, may embody pore-forming substances. Nitric acid has a benefit of being a passivating agent, for example, for use with orthopedic implant embodiments, for example, with CPT as the metal or metal alloy.

Thus, the porous material may be made by providing the metal or metal alloy, and the pore-holding substance; providing a thermal sprayer; mixing the metal or metal alloy, and the pore-holding substance to provide as first intermediate a thermal spray mixture, and providing the thermal spray mixture to the thermal sprayer; thermally spraying the thermal spray mixture with the thermal sprayer to provide the second intermediate, for example, as a coating ; and contacting the second intermediate with the pore-forming substance to make the porous material. The porous material may be a porous coating. The coating may be on an orthopedic implant.

In the methodology herein, in general, conditions are those sufficient to make the first intermediate; the second intermediate; and/or the porous material. Additional step(s) may be conducted thereto and therewith to make further product(s) therefrom.

Accordingly, for example, the pores can be used to contain medicaments, vitamins, tracing isotopes, poisons, and so forth. These may be released subsequently, for instance, upon or subsequent to implantation. In addition, a hydroxyapatite coating may be sprayed on a porous material to further engender bone growth.

The following example further illustrates the invention.

### EXAMPLE

For each run, a thermal spray chamber with a three-angle robotic electric thermal spray head is evacuated to about 50 Torr, and then filled with Argon gas to about 760 Torr. Two titanium tabs are rotated on the periphery of a rotating turntable under the spray head and thermally sprayed with CPT fed in powder form (75˜175 um) from a Praxair Model 1264 powder feeder to a depth of about 0.005 inches to form a bond coat. A mixture of the CPT powder as metal or metal alloy and apatitic abrasive, a hard, granular, multi-phase calcium phosphate powder from Himed, as pore-holding substance simultaneously fed from another Praxair Model 1264 powder feed is thermally sprayed onto the bond coat of the tabs, as follows:

| | Pore-holding | Metal or metal |
|---|---|---|
| Run # | substance size | alloy:pore-holding substance ratio |
| 1 | 63~106 um | 1:1 |
| 2 | 63~106 um | 5:2 |
| 3 | -180 um | 5:2 |

to about 0.010-inch on top of the corresponding bond coat to make second intermediates. Thus, an about 0.015-inch coating is applied.

The second intermediates from each run are cooled, and one from each run is set aside. The other second intermediates from each run are briefly sprayed with abrasive to remove any loose material, immersed in room temperature aqueous nitric acid for half an hour, rinsed with water, and dried. The nitric acid treated samples from each run are visually inspected with the naked eye and inspected under a stereo microscope, and compared to the second intermediate samples set aside and not treated with nitric acid. At least the apatitic abrasive containing second intermediate from run #3 appears to present grains of calcium phosphate. Each of the nitric acid treated samples is a porous material, and has significantly more porosity than the corresponding second intermediate.

Under the stereo microscope, the porous materials are compared to each other. The extent of porosity, and satisfaction as potentially viable candidates for orthopedic implant coatings, increases in order from run #1, to run #2, to run #3.

### CONCLUSION TO THE INVENTION

The present invention is thus provided. Various feature(s), part(s), step(s), subcombination(s) and/or combination(s) may be employed with or without reference to other feature(s), part(s), step(s), subcombination(s) and/or combination(s) in the practice of the invention, and numerous adaptations and modifications can be effected within its spirit, the literal claim scope of which is particularly pointed out as follows:

What is claimed is:

1. A method for making a porous material made with a metal or metal alloy, which embraces a constituent of the metal or metal alloy, and which has a suitable pore size and distribution, and interconnecting porosity, said method comprising the following steps:
providing a proximal intermediate of a matrix of the metal or metal alloy, in which is dispersed a pore-holding substance; and
contacting the proximal intermediate with a pore-forming substance under conditions such that at least some of the pore-holding substance in the matrix contacted with the pore-forming substance is reduced in size or removed to leave pores in remaining metal or metal alloy to provide the constituent of a metal or metal alloy with pores.

2. The method of claim 1, wherein the proximal intermediate is provided from an initial intermediate, by steps further comprising providing the metal or metal alloy, and the pore-holding substance; combining the metal or metal alloy, and the pore-holding substance to form the initial intermediate; and forming from the initial intermediate, the matrix of the metal or metal alloy, in which is dispersed the pore-holding substance.

3. The method of claim 2, wherein the forming step includes thermal spraying.

4. The method of claim 3, wherein the thermal spraying is conducted under sub-atmospheric conditions.

5. The method of claim 3, wherein the initial intermediate includes the metal or metal alloy in powdered form, and the pore-holding substance in powdered form.

6. The method of claim 3, wherein the initial intermediate is in a form of a cored wire with the metal or metal alloy forming a sheath around the pore-holding substance, with the pore-holding substance in powdered form.

7. The method of claim 1, wherein the pore-forming substance is an acid, a base, or other reactive compound or composition.

8. The method of claim 7, wherein the pore-forming substance is also a passivating agent.

9. The method of claim 1, wherein the constituent of a metal or metal alloy with pores is a coating on a substrate.

10. The method of claim 9, wherein the coating and substrate together form an orthopedic implant.

11. The method of claim 1, wherein the metal is aluminum, chromium, cobalt, iron, nickel, tantalum, titanium, vanadium, zirconium, gold, silver or platinum; the metal alloy includes cobalt, chromium, nickel, iron, molybdenum titanium, aluminum or vanadium; the pore-holding substance is a calcium phosphate, a dicalcium phosphate, a tricalcium phosphate, or a magnesium oxide; and the pore-forming substance is an acid.

12. The method of claim 11, wherein the constituent of a metal or metal alloy with pores is a coating on a substrate; the coating and substrate together form an orthopedic implant; the metal or metal alloy is or includes titanium; and the acid is nitric acid.

13. The method of claim 12, wherein the pores formed have a volume porosity about from 30 to 70 percent, an average pore size about from 100 to 1000 microns, and a thickness of about from 500 to 1500 microns.

14. An intermediate for making a porous material, said intermediate comprising at least one of the following (A, B):
(A) a first intermediate for making the porous material, which embraces a metal or metal alloy, and a pore-holding substance, wherein at least the pore-holding substance is in a form of a powder, and the first intermediate can be employed to make a second intermediate for the porous material; and
(B) the second intermediate for the porous material, which embraces a matrix of the metal or metal alloy, in which is dispersed the pore-holding substance, wherein the second intermediate can be employed to make the porous material by removing or reducing in size the pore-holding substance in said matrix.

15. The intermediate of claim 14, which is the first intermediate, wherein the metal or metal alloy is also in a form of a powder.

16. The intermediate of claim 15, wherein the metal or metal alloy, and the pore-holding substance are provided, independently at each occurrence, from the powder sizes or powder size samples as follows:
the metal or metal alloy: about from 35 to 500 um (+35/−400 mesh);
about from 44 to 150 um (+100/−325 mesh); and/or
about from 75 to 175 um (+80/−200 mesh); and
the pore-holding substance: about from 35 to 500 um (+35/−400 mesh);
about from 53 to 350 um (+45/−270 mesh);
about from 63 to 106 um (+140/−230 mesh);
about from 150 um to 200 um (+70/−100 mesh);
about from 180 to 360 um (+45/−80 mesh);
about from 180 to 300 um (+50/−80 mesh); and/or
about 180 um or less (+80 mesh).

17. The intermediate of claim 14, which is the first intermediate, wherein the metal or metal alloy is in a form of a sheath for a cored wire, with the pore-holding substance forming a core of the wire.

18. The intermediate of claim 14, wherein the metal is aluminum, chromium, cobalt, iron, nickel, tantalum, titanium, vanadium, zirconium, gold, silver or platinum; the metal alloy includes cobalt, chromium, nickel, iron, molybdenum titanium, aluminum or vanadium; and the pore-holding substance is a calcium phosphate, a dicalcium phosphate, a tricalcium phosphate, or a magnesium oxide.

19. The intermediate of claim 18, wherein the metal or metal alloy is or includes titanium.

20. A porous material made with a metal or metal alloy, which comprises a constituent of the metal or metal alloy, which has a suitable pore size and distribution, interconnecting porosity, and other features to distinguish it from prior art—optionally which is made from a method embracing steps of providing a proximal intermediate of a matrix of the metal or metal alloy, in which is dispersed a pore-holding substance; and contacting the proximal intermediate with a pore-forming substance under conditions such that at least some of the pore-holding substance in the matrix contacted with the pore-forming substance is reduced in size or removed to leave pores in remaining metal or metal alloy to provide the constituent of a metal or metal alloy with pores.
